(19) United States
(12) Patent Application Publication
(10) Pub. No.: US 2003/0133828 A1
(43) Pub. Date: Jul. 17, 2003

(54) Method for reducing airborne biological agents while processing mail

(75) Inventor: Andrew T. Lucas, Broken Arrow, OK (US)

Correspondence Address: PERKINS, SMITH & COHEN LLP, ONE BEACON STREET, 30TH FLOOR, BOSTON, MA, 02108, US

(73) Assignee: Lockheed Martin Corporation

(21) Appl. No.: 10/331,830

(22) Filed: Dec. 30, 2002

Related U.S. Application Data
(60) Provisional application No. 60/344,844, filed on Dec. 31, 2001.

(51) Int. Cl. A61L 2/00; A61L 9/00; A62B 7/08; B01J 19/08

(52) U.S. Cl. 422/22; 422/1; 422/24; 422/28; 422/32; 422/123; 422/186; 422/186.05; 422/300; 422/305; 422/307

(57) ABSTRACT

Methods to reduce the volitation and for neutralization of biological agents contained and emanating from objects such as mail pieces include the steps of providing an electrical charge to the biological agents, rendering the electrically charged airborne biological agents stationary, and neutralizing the stationary biological agents and the less volitant biological agents. The systems to reduce the volitation and for neutralization of biological agents include components capable of effecting the method described above.

### CROSS REFERENCE TO RELATED APPLICATIONS

This application claims priority of U.S. Provisional Application No. 60/344,844 filed on Dec. 31, 2001, which is herein incorporated by reference.

### BACKGROUND OF THE INVENTION

This invention relates generally to the neutralization or sterilization of hazardous material where the hazardous material is transported via the mail and, more particularly, to reduce volitation of the hazardous material and to the neutralization of a biological agent disposed in a mail piece where the biological agent has been rendered easily airborne by removing electrical charge from the biological agent.

The possibility of using postal services or delivery services to release biological agents has been demonstrated. Unsuspecting recipients can be harmed by the release of such biological agents. Equally at risk are those handling the mail pieces or the packages to be delivered. One procedure to safeguard against the release of biological agents is to irradiate all the mail pieces or packages with a strong enough dosage to render the biological agents inert. Such a procedure may not be completely reliable. Additional safeguards would be prudent to lower the risks of exposure and to increase the probability of contaminant detection.

Some of the biological agents are rendered easily airborne by removing electrical charge from the biological agent. There is a need for a system that restores the electrical charge to the biological agents and renders the biological agents less airborne (reduces their volitation).

### SUMMARY OF THE INVENTION

The present invention discloses systems and methods to reduce the volitation of the biological agents in order to hold such biological agents stationary so that they can be sterilized or their affects otherwise neutralized. The method to reduce the volitation and for neutralizing the biological agents includes:

A. restoring the electrical charge to the biological agents thereby reducing the volitation of the biological agents, limiting airborne exposure;

B. rendering the electrically charged biological agents stationary by means of attraction to an alternatively charged system;

C. neutralizing the stationary biological agents and the less volitant biological agents;

The system to implement the method of this invention includes a transport sub-system for transporting the mail pieces on a conveyor, means for restoring an electrical charge to the biological agents contained in and emanating from mail pieces, and means for rendering the electrically charged airborne biological agents substantially stationary. Embodiments of means for restoring the electrical charge to the biological agents include, but are not limited to, charging plates (or electrodes) and ionizing radiation such as X-rays and UV. Embodiments of means for rendering the electrically charged airborne biological agents substantially stationary include, but are not limited to, charging plates and charged objects. Once the biological agents are substantially stationary, the biological agents can be rendered harmless by sterilization utilizing means such as UV radiation. (The rendering of the biological agents harmless by sterilization is also referred to herein as neutralizing.) For a better understanding of the present invention, reference is made to the accompanying drawings and detailed description.

### BRIEF DESCRIPTION OF THE DRAWINGS

FIG. 1 is a flow chart representative of the methodology of the charging and sterilization system of the present invention;

FIG. 2 pictorially and schematically represents one embodiment of the system of the present invention;

FIG. 3 depicts a graphical schematic representation of one embodiment of the system for restoring the charge to the biological agents;

FIG. 4 depicts a graphical schematic representation of an embodiment of the system to charge the mail piece; and,

FIG. 5 depicts a schematic graphical representation of another embodiment of the system to charge the mail piece.

### DETAILED DESCRIPTION OF THE EMBODIMENTS

System and methods to render biological agents less airborne (reduce their volitation) and provide for the neutralization of the biological agents are disclosed below.

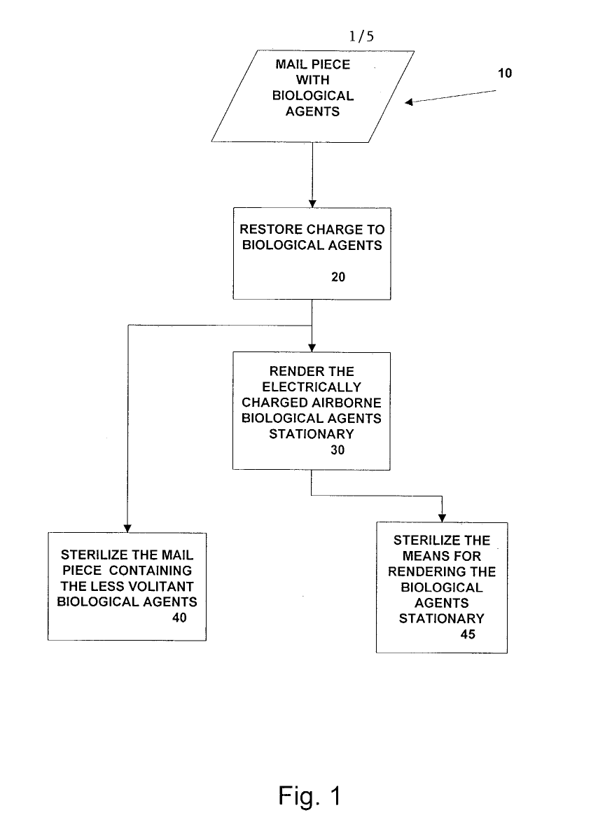

A flow chart of an embodiment of a method for reducing the volitation of airborne biological agents where the biological agents arise from containers 10, such as envelopes or mail pieces, is depicted in FIG. 1. Starting from mail piece 10 containing biological agents and airborne biological agents, where the biological agents have been rendered easily airborne by removing their electrical charge, charge is restored to the both the airborne biological agents and the biological agents in the mail piece (step 20, FIG. 1). The electrically charged airborne biological agents are, then, rendered stationary (step 30, FIG. 1). Finally, both the mail pieces 10 and the means for rendering the biological agents stationary are sterilized (steps 40, 45, FIG. 1). The process described above can take place in an enclosed or contained section of a system for transporting the containers. If the process takes place in an enclosed volume of space, the ambient air contained in that enclosed volume of space can be further filtered utilizing a conventional electrostatic filtering system in addition to or instead of other filtering systems.

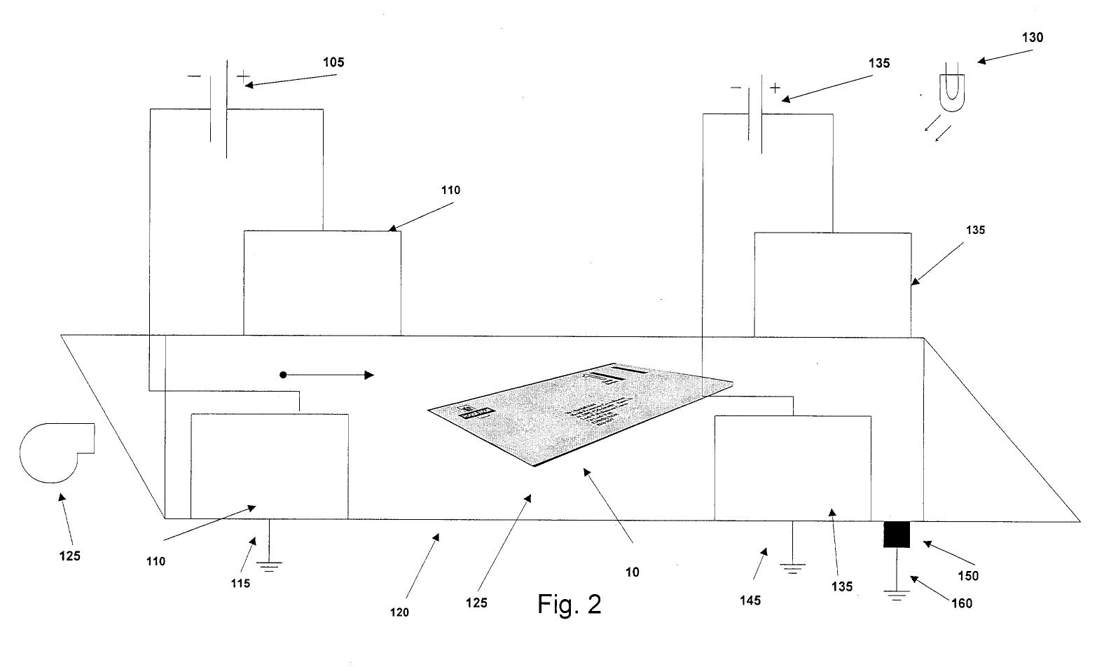

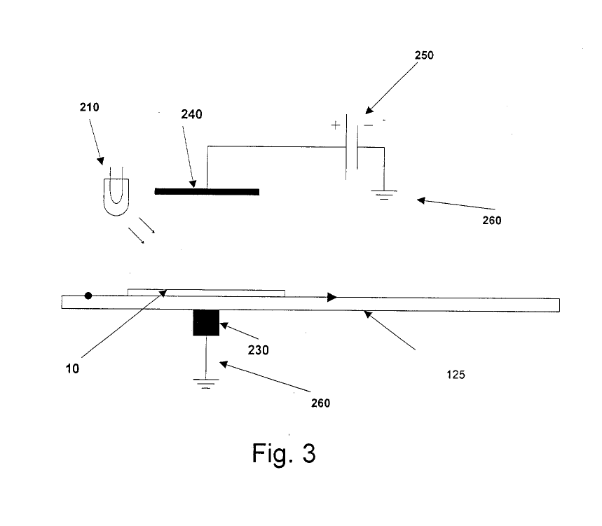

One embodiment of a system that performs the above described process is depicted in FIG. 2. A transport system 120 delivers the mail piece 10, which may contain biological agents, to the vicinity of the charging system 115. (The term “mail piece” as used herein includes a container, a package in a delivery system or any item in a delivery system which could be used to surreptitiously deliver biological agents.) Transport systems, such as conveyor belts and means for driving the conveyor belts are used in package delivery operations and are known in the art. An air flow system 125, such as a fan or blowers (schematically shown by a fan symbol), also assists in delivering the airborne biological agents to the vicinity of charging system 115. The parallel plates 110 and the voltage source 105 constitute an embodiment of the charging system 115. While the placement of the electrodes 110 are as shown in FIG. 2 generates an electric field substantially parallel to the plane of the transport web 125 and substantially perpendicular to the direction of transport, an electric field substantially perpendicular to be plane of the web 125 can be generated by the configuration shown in FIG. 3. Referring to FIG. 3, electrodes 230 and 240 when connected to voltage source 250 produce an electric field substantially perpendicular to the surface of the transport the web 125. In embodiments of charging systems, it is likely that a timing system is included in the charging means. A sensor (not shown), photo-electric or mechanical, detects the time at which the mail piece 10 enters the region in which charging will occur. The charging means are energized upon receiving a signal from the sensor indicating a mail piece 10. Similarly the charging means could be de-energized when all mail pieces (or when one mail piece) leaves the region.

In the embodiments utilizing electrodes and a voltage source, the voltage and distance between the electrodes (or the geometry of the electrodes) is selected so that the biological agents are charged or ionized but such that avalanche breakdown of the ambient gas does not occur. Such designs are known in the art (see, for example, S. C. Brown, Basic Data of Plasma Physics, M.I.T. Press, Cambridge, Mass., 1961 pp. 268-274). The actual voltage required is determined by the shape of the electrode, the distance between electrodes, the composition.(elements and density) of the ambient gas. Typical voltages are in the kilovolt range. A brush type electrode 150 is placed in contact with the transport web 125 and connected to ground 160. The grounding electrode 150 provides means for the discharging the transport web 125. The electrically charged airborne biological agents are rendered stationary by an electrical holding or collection system 145. The parallel plates 140 and the voltage source 135 constitute an embodiment of the electrical holding system 145. Sources of radiation 130, such as UV-C (Ultra Violet-C) radiation, constitute neutralizing means. As used herein, the term “neutralizing” refers to deactivating, degrading, rendering substantially harmless, decontaminating, and/or sterilizing any hazardous agent detected.

In another embodiment of the charging system 115, ionizing radiation is utilized to restore the charge. Referring to FIG. 3, ionizing radiation from source 210 constitutes an embodiment of the charging system that includes a source of ionizing radiation. Possible embodiments of sources of ionizing radiation are sources of soft X-rays, X-rays, ultraviolet radiation. Soft X-ray sources, with wavelength between 10⁻¹⁰ and 10⁻⁹ meters, have been used successfully in the charging of photoconductors (see, for example, U.S. Pat. No. 6,058,003 to M. Hirano et al. issued on May 2, 2000), and are capable of penetrating envelopes. The actual wavelength spectrum required will be determined by photo-ionization cross section of the biological agents. The wavelengths of interest are typically in the range from below 200 nanometers (nm) to tenths of nanometers (energetic UV to soft X-ray). The X-ray wavelengths are typically selected by the availability of sources and the efficiency of ionization for a specific range of possible biological agents.

In yet another embodiment of the charging system 115, both ionizing radiation and the application of voltage are utilized to restore the charge. In one configuration, shown in FIG. 3, electrodes 230 and 240 produce an electric field approximately perpendicular to the transport web 125. The voltage 250 is chosen to prevent avalanche breakdown of the ambient air in the presence of the ionizing radiation. In the configuration shown, the transport web 125 is grounded by electrode 230. The polarity of the voltage can be varied from that of FIG. 3.

The same electrodes (or charging plates) that constitute the charging means, in those embodiments utilizing electrodes in the charging means, can also serve as holding means since charged airborne particles will be attracted to one of such electrodes.

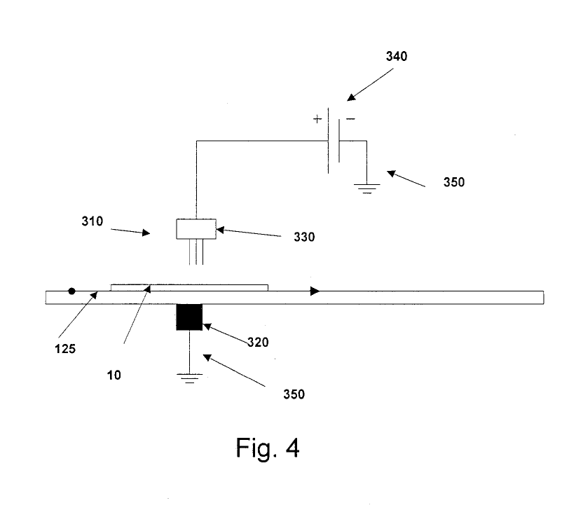

In another embodiment of the electrical holding system, shown in FIG. 4, corona charging system 310 is utilized to charge mail piece 10 with the opposite charge to that which will be restored to the airborne particles. Corona charging system 310 includes corona electrode 330, the electrode 320 and voltage source 340. Corona charging of dielectrics has been used in xerography and is also in web coating technology (see, for example, U.S. Pat. No. 3,254,215 to K. M. Oliphant issued on May 31, 1966 and U.S. Pat. No. 6,399,151 to Zaretsky issued on Jun. 4, 2002). When the charge is restored to airborne particles, the airborne charged particles are attracted to the charged mail piece 10. This process can be aided by applying an electric field substantially perpendicular to the transport web 125, subsequent to the charging of the mail piece 10 and the restoring of the charge to the airborne particles.

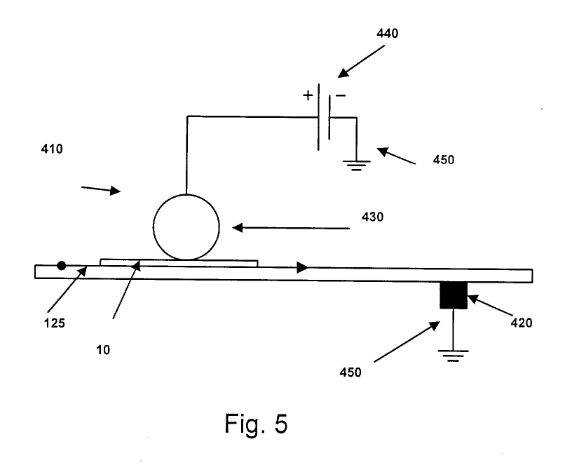

In yet another embodiment of the electrical holding system, shown in FIG. 5, contact (triboelectric) charging system 410 is utilized to charge mail piece 10 with the opposite charge to that which will be restored to the airborne particles. Charging system 410 includes a charging roller 430 electrically connected to voltage source 440 which also connected to ground 450. Grounding connector 420 could be the same as grounding connector 160 in FIG. 2. The charging roller 430 should retain charge and deform slightly on contact to insure good electrical contact without damaging the mail piece 10. A roller consisting of an inner conductor such as a metal, a slightly deformable (elastic) material of intermediate conductivity and an outer layer of a poorer conductor (better insulator). For example, a roller such as that described in U.S. Pat. No. 3,702,482 to C. Dolcimascolo et al. issued to Nov. 7, 1972 could be used. The use of a charging roller or blade requires contact and therefore, requires consideration of prevention of damage to the mail piece 10. Several means are available to prevent damage to the mail piece 10. The contact roller can be spring loaded so contact is maintained with controlled contact pressure. Alternatively, the contact roller can be placed in contact with the mail piece by means of a solenoid and damper system. This alternative would require sensing the arrival and exit of the mail piece 10 from the contact area. Another mechanism is to use the back pinch belt of the transport to maintain the contact pressure. Using the travel belts to maintain pressure points is a common application in transport design. However, a contact charging system requires a lower voltage than a corona charging system.

One embodiment of the neutralizing means includes one or more sources of UV radiation of wavelength below 290 nm (UV-C radiation). If possible, the spectrum of the UV-C radiation should be tailored so that the spectrum has a high content of radiation at 260 nm since such a spectrum would optimize the germicidal effect.

Another embodiment of the neutralizing means includes electrodes, utilized as holding means, coated with a photocatalyst and illuminated by radiation with a wavelength equal to the band gap of the photocatalyst. The relative humidity of ambient would probably need to be maintained in a given range for the photocatalyst to be effective (see, for example, U.S. Pat. No. 5,993,738 to Goswani issued on Nov. 30, 1999).

It should be apparent that other charging means and neutralizing means can be used and that the charging system and holding system may be combined. If triboelectric charging means are used, the charging system should also be sterilized after exposure to the biological agents. If the above described system is located in an enclosed volume of space, the volume of ambient could be evacuated or the ambient pressure reduced so that ionizing radiation from the neutralizing means 130 does not cause ambient air breakdown between the holding plates 140.

While the above embodiment utilizes UV radiation, it should be noted that other neutralizing means such as other ionizing radiation (X-rays, for example) can be used. It should also be noted that, while the term biological agents was used above, the system and method can be applied to other particulates.

Although the invention has been described with respect to various embodiments, it should be realized that this invention is also capable of a wide variety of further and other embodiments all within the spirit and scope of this invention.

That which is claimed is:

1. A method for reducing airborne biological agents from mail pieces, said method comprising the steps of:
providing an electrical charge to the biological agents, the biological agents originating from the mail pieces;
rendering the electrically charged airborne biological agents substantially stationary.

2. The method of claim 1 wherein the step of providing the charge to the biological agents further comprises the steps of:
transporting the mail piece along a transport direction;
applying an electric field.

3. The method of claim 1 wherein the step of providing the charge to the biological agents further comprises the steps of:
transporting the mail piece along a transport direction;
illuminating an area traversed by the mail piece with ionizing radiation.

4. The method of claim 1 wherein the step of rendering the electrically charged airborne biological agents stationary further comprises the steps of:
transporting the mail piece along a transport direction;
applying an electric field in a direction substantially perpendicular to the transport direction.

5. The method of claim 2 wherein the step of rendering the electrically charged airborne biological agents stationary further comprises the step of applying an additional electric field in a direction substantially perpendicular to the transport direction.

6. The method of claim 1 further comprising the steps of:
neutralizing the biological agents rendered stationary; and,
neutralizing the biological agents contained in the mail pieces.

7. The method of claim 6 wherein the step of neutralizing the biological agents further comprises the step of applying UV-C radiation to the biological agents.

8. A system for reducing airborne biological agents from mail pieces comprising:
a transport sub-system for transporting the mail pieces along a transport direction;
means for providing an electrical charge to the biological agents originating from mail pieces; and,
means for rendering the electrically charged airborne biological agents substantially stationary.

9. The system of claim 8 wherein the means for providing an electrical charge to the biological agents further comprise means for applying an electric field.

10. The system of claim 8 wherein the means for providing an electrical charge to the biological agents further comprise a source of ionizing radiation illuminating an area traversed by the mail piece.

11. The system of claim 8 wherein the means for rendering the electrically charged airborne biological agents stationary further comprise means for applying an electric field in a direction substantially perpendicular to the transport direction.

12. The system of claim 8 further comprising:
means for neutralizing the biological agents rendered stationary.

13. The system of claim 8 further comprising:
means for neutralizing the biological agents contained in the mail pieces.

14. The system of claim 12 wherein the neutralizing means further comprise means for applying UV-C radiation.

15. The system of claim 13 wherein the neutralizing means further comprise means for applying UV-C radiation.
